(19) United States
(12) Patent Application Publication
(10) Pub. No.: US 2021/0057060 A1
(43) Pub. Date: Feb. 25, 2021

(54) SYSTEMS AND METHODS FOR USING DATABASES, DATA STRUCTURES, AND DATA PROTOCOLS TO EXECUTE A TRANSACTION IN A DATA MARKETPLACE

(71) Applicant: Universal Research Solutions, LLC, Columbia, MO (US)

(72) Inventor: Ali Adel Hussam, Columbia, MO (US)

(21) Appl. No.: 16/989,692

(22) Filed: Aug. 10, 2020

Related U.S. Application Data
(60) Provisional application No. 62/885,188, filed on Aug. 9, 2019.

(51) Int. Cl. G16H 10/60; G16H 15/00; G06F 16/22

(52) U.S. Cl. CPC G16H 10/60; G06F 16/2272; G16H 15/00

(57) ABSTRACT

Methods, systems, apparatus, and computer programs, for executing a transaction in a data marketplace. In one aspect, the method includes receiving, from a user device, a first data structure that includes one or more fields structuring first data that represents a request for patient information, identifying one or more second data structures in a patient registry database that satisfy the request for patient information that is represented by the first data, determining a level of compensation for patients that correspond to the one or more second data structures, and executing programmed logic structured by fields of a fourth data structure that causes a data marketplace computer to provide compensation to a wallet of each of the particular patients that correspond to the at least one or more of the identified second data structures from which at least a portion of the second data was selected.

### CROSS-REFERENCE TO RELATED APPLICATIONS

This application claims the benefit of U.S. Provisional Patent Application No. 62/885,188 filed Aug. 9, 2019. The entire contents of which is hereby incorporated by reference in their entireties.

### BACKGROUND

Medical forms are used to collect information regarding a patient's symptoms and conditions. One technique for preparing a medical form is to manually edit a pre-existing form (e.g., a form existing in Microsoft Word™ format) with new or customized questions. The form is then sent to review boards for review through a physical or electronic mailing. Additionally, once a form has been finalized, it may be presented to a patient, study participant or other individual (collectively referred to as “patients” herein, without limitation, for purposes of convenience). For example, physicians may present patients with the forms when the patient visits the physician's office. Additionally, hardcopy (i.e., paper) versions of medical forms may be distributed to patients for completion. For patients who have not completed medical forms prior to the patient's examination, the patient may often complete the medical form at the physician's office by filling out a hardcopy of the form.

Frequently, the patient's responses to the questions included in the medical forms are entered into a computerized system by medical personnel. In this case, in order for a physician to review the patient's responses, the physician may access the computerized system and view the answers to the questions, which is often a lengthy process of reviewing individual questions.

### SUMMARY

According to one innovative aspect of the present disclosure, a system for executing a transaction in a data marketplace is disclosed. The system can include one or more data processing apparatus and one or more computer-readable storage devices having stored thereon instructions that, when executed by the one or more data processing apparatus, cause the one or more data processing apparatus to perform operations. In one aspect, the operations include receiving, by one or more computers of a data marketplace and from a user device, a first data structure that includes one or more fields structuring first data that represents a request for patient information, identifying, based on the receipt of the first data that represents the request for patient information, one or more second data structures in a patient registry database that satisfy the request for patient information that is represented by the first data, wherein each of the one or more second data structures (i) correspond to a particular patient and (ii) include fields structuring second data that represents patient information for the particular patient, determining, by the one or more computers of the data marketplace, a level of compensation for each of the particular patients, based on an evaluation of third data structured by fields of a plurality of third data structures, wherein each third data structure includes fields structuring data representing a patient profile for one of the particular patients, and based on a determination, by the one or more computers of the data marketplace that a subsequent request from the user device selects at least a portion of the second data of at least one of the one or more identified second data structures, executing programmed logic structured by fields of a fourth data structure that causes the one or more computers to provide compensation to a wallet of each of the particular patients that correspond to the at least one or more of the identified second data structures from which at least a portion of the second data was selected, wherein the provided compensation is based on the determined level of compensation.

Other aspects include corresponding methods, apparatus, and computer programs to perform actions of methods defined by instructions encoded on computer storage devices.

These and other versions may optionally include one or more of the following features. For example, in some implementations, the operations can further include generating, by the one or more computers of the data marketplace, a fifth data structure that includes fields structuring rendering data that, when executed by the user device, causes the user device to display a graphical record, for each particular second data structures of the one or more identified second data structures, wherein the graphical record displays at least a portion of the patient information for the particular patient that corresponds to the particular second data structure and providing, by the one or more computers of the data marketplace, the fifth data structure that includes the fields structuring the rendering data to the user device.

In some implementations, receiving, by one or more computers of a data marketplace and from a user device, a sixth data structure that includes one or more fields structuring second data that represents a user's selection of one or more of the graphical record displays that each corresponds to one of the identified second data structures.

In some implementations, the patient information can include health records, medical instrument information, or outcome information.

In some implementations, the operations can further include generating, by the one or more computers of the data marketplace, a report that summarizes the second data of at least one of the one or more identified second data structures that correspond to the one or more second data structures having second data that was selected by the user and providing, by the user one or more computers, the generated report to the user without providing the second data of the one or more identified second data structures.

In some implementations, the data representing a patient profile for one of the particular patients can include one or more of: (i) data representing a score indicating quality of the patient information, (ii) data that describes a form of compensation for the patient, (iii) data that describes a patient's level of compliance (iv) data describing a volume of patient surgeries, (v) data describing procedure types that were performed on the patient, (vi) data describing patient reported outcome measures (PROMs) used, (vii) type of data stored in the patient registry database for the patient, (viii) an amount of data provided to the patient registry database by the patient, and (ix) a utility score indicating a usefulness of the data stored in the patient database registry for the patient.

In some implementations, identifying, based on the receipt of the first data that represents the request for patient information, one or more second data structures in a patient registry database that satisfy the request for patient information that is represented by the first data, wherein each of the one or more second data structures (i) correspond to a particular patient and (ii) include fields structuring data that represents patient information for the particular patient can include obtaining, by the one or more computers of the data marketplace, the first data from the first data structure, generating, by the one or more computers of the data marketplace, a search query that includes the obtained first data, and executing, by the one or more computers of the data marketplace, the generated search query against the patient registry database.

These and other aspects of the present disclosure are further described in the detailed description, the drawings, and in the claims.

### BRIEF DESCRIPTION OF THE DRAWINGS

FIG. 1 is a contextual diagram of an example of a system for using databases, data structures, and data protocols to execute an electronic transaction in a data marketplace.

FIG. 2 is a flowchart of an example of a process for using databases, data structures, and data protocols to execute an electronic transaction in a data marketplace.

FIG. 3 is a block diagram of system components that can be used to implement a system for using databases, data structures, and data protocols to execute an electronic transaction in a data marketplace.

### DETAILED DESCRIPTION

The present disclosure is directed towards systems, methods, and computer programs, for implementing anonymizing and monetizing patient data. Business entities such as online advertisers can use collected data to make inferences about an entity such as a type of content an entity may want to consume, a type of item an entity may want to purchase, a trait describing an entity, or the like. However, such business entities do not enable the entity whose data was used to be compensated for the use of such data.

The present disclosure addresses these, and other, problems by providing a system that includes, inter alia, processes for de-identifying aggregated entity data to create obfuscated entity data, making the obfuscated data searchable to one or more information consumers, determining if the one or more information consumers has accessed the obfuscated data, and based on a determination that the one or more information consumers has accessed the obfuscated data, providing compensation to an account of an entity that is associated with the obfuscated data that was accessed. In some implementations, an account can include, for example, a traditional bank account. In some implementations, the account can include a digital wallet. However, these types of accounts should not be viewed as limiting, as an account can be anything that is capable of receiving, transferring, storing, or tracking units of value associated with an entity. Units of value can include, for example, a currency units (e.g., dollars, euros, or the like), a cryptocurrency (e.g., BTC, ETH, or the like), loyalty rewards (e.g., rewards points, shopper points, hotel points, frequent flyer points, hospital points, or the like), or the like.

An entity is associated with obfuscated data if the obfuscated data describes or is related to the entity. Data that describes, or is related to the entity can include information such as the entity's medical records. However, the data that describes, or is related to, the entity can include any data related to the entity including, but not limited to, for example, web browsing activity, shopping activity (e.g., purchase activity), mobile application usage information, mobile device usage information, sales activity, inventory information, or the like.

Accordingly, the present disclosure provides solutions to the problem of compensating an entity for the use of their obfuscated data in a data marketplace. The solution to this problem can be achieved by using the present disclosure to map obfuscated data accessed by a data consumer via a data market place to a particular entity. The present disclosure can determine an account associated with the particular entity. Then, the present disclosure can compensate the particular entity for the use of the entity's data. For purposes of the present disclosure, an entity can be a person, an object (e.g., a product or line of products), a service or line of services, a business, an organization, or the like.

Accordingly, in some implementations, the entity can be a person that is or was a patient and the de-identified information can be patient information related to the patient such as the patient's medical records. However, the present disclosure need not be so limited. Instead, the entity can be any person, object, service, business, or organization and the de-identified information can be any information related to the person object, service, business, or organization. An example of the present disclosure using patients and patient information is described below.

In some implementations, the present disclosure can provide a data marketplace for sharing data describing patient information related to a patient with one or more data vendors (“vendors”) in such a manner that the patient's actual data is not shared with the one or more vendors. Actual data can include information or data related to an entity that has not been de-identified. By way of example, actual data can include an entity's medical records that include sensitive and protected information such as information protected by the Health Insurance Portability and Accountability Act (HIPAA). However, the present disclosure can be any data that exists in a first state, where the first state is a state that exists prior to a de-identified data state, with the de-identified data state being a second state.

The present disclosure can include compensating functionality that is configured to compensate a patient based on a determination that information describing the patient's information is shared with a vendor. In some implementations, sharing data describing a patient's information with a vendor can include selling one or more rights to access, review, use, monetize, modify, or any combination thereof, data describing the patient's information by the vendor. In some implementation, data describing the patient's information can be provided to the vendor responsive to a request for such information by the vendor.

Data describing patient information related to one or more patients can be referred to in this specification as obfuscated data or obfuscated patient information. Obfuscated patient information is data that substantively captures the patient's actual information without providing the patient's actual patient information. Such patient information may include information that is sensitive, private, or a combination thereof such as the patient's medical records, or a portion thereof. Patient information can be obtained from one or more patient devices, one or more medical service provider devices, one or more other devices, or a combination thereof. In some implementations, a device can be a wearable device or a non-wearable device. By way of example, a device can include any type of computer including, but not limited, a personal fitness device, a smartwatch, a smartphone, a tablet, a laptop computer, a desktop computer, a server, multiple servers, or the like.

The present disclosure can compensate a particular patient based on determinations that a vendor has requested, selected, accessed, viewed, purchased, or any combination thereof, obfuscated patient information that is based on the particular patient's patient information. In some implementations, a level of compensation for a patient can be determined based on an evaluation of a patient profile. In such implementations, if obfuscated patient information for a particular patient is requested, selected, accessed, viewed, purchased, or any combination thereof, by a vendor, compensation can be provided to an account such as a digital wallet of that particular patient based on a variety of factors established in a patient profile for the particular patient.

Accordingly, these aspects of the present disclosure described above, when compared to conventional systems, can provide increased data security while also providing means for enhancing the ability of a system to gather more patient information through use of financial incentives. That is, a patient may be increasingly inclined to share the patient's information if the patient knows that patient is going to be compensated for use of the patient's information. This specification uses the terms “information” and “data”. For purposes of this specification the terms “information” and “data” can be used interchangeably to mean a representation of any knowledge, facts, details, attribute, or the like, related to a subject, entity, person, place, object, thing, or the like.

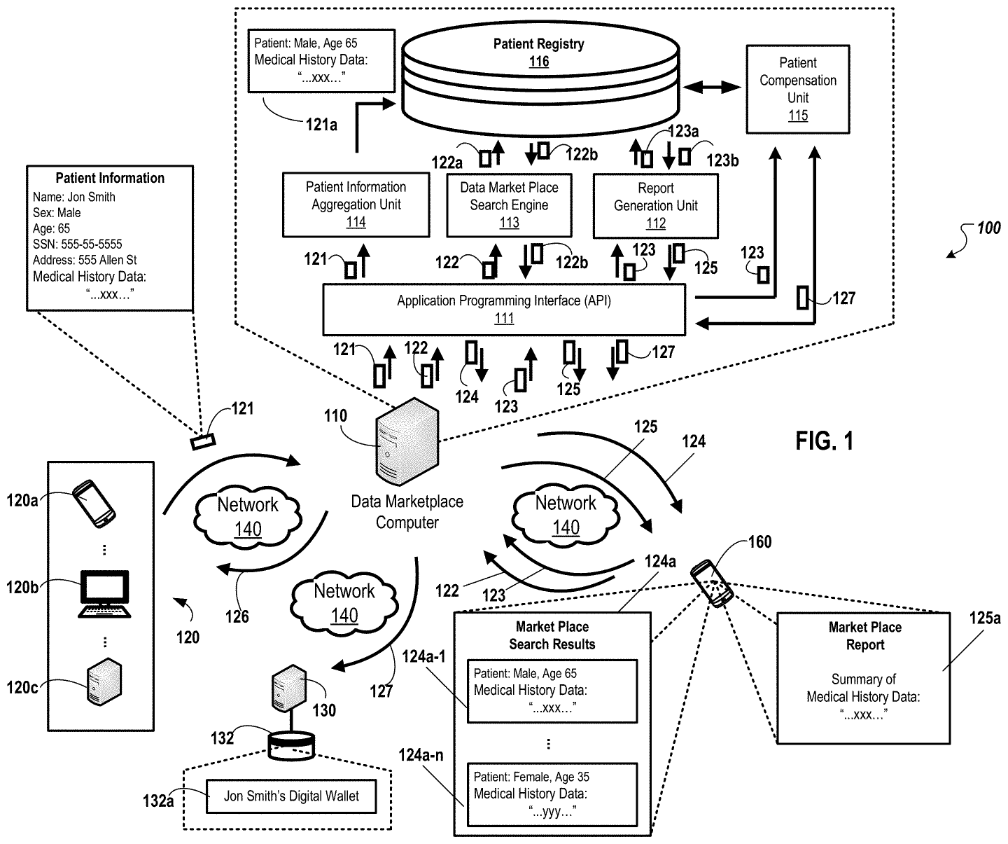

FIG. 1 is a contextual diagram of an example of a system 100 for using databases, data structures, and data protocols to execute an electronic transaction in a data marketplace. The system includes a data marketplace computer 110, one or more patient devices 120, a computer 130 storing a patient account such as digital wallet 132a, a network 140, and a vendor device 160. The one or more devices 120, the computer 130, and the vendor device 160 are communicatively coupled to, and can communicate with, the data marketplace computer 110 through the network 140.

The network 140 can include one or more of a local area network (LAN), a wide area network (WAN), a wired network, or a wireless network. A wired network can include an ethernet connected network, fiber optic connected network, or the like. A wireless network can include a WI-FI network, a cellular network, or the like. In some implementations, the network 140 can include the Internet. In some implementations, the one or more devices 120 can include a mobile device such as a smartphone, a tablet computer, a personal digital assistant, or the like. In some implementations, the one or more devices 120 can include a desktop computer. In some implementations, the one or more patient devices 120 can include a database such as a cloud-based computing environment.

The one or more devices 120 can transmit patient information 121 over the network 140. Patient information 121 can include data describing one or more attributes of a patient. The one or more attributes can include any attribute that is related to a patient. By way of example, the patient information can include one or more of name, age, sex, social security number, address, or medical history data. The medical history data can include one or more of health records, patient reported outcome measures (“PROMs”), medical study instrument information, outcome information, disease history, treatment history, and pharmaceutical drug use history. A PROM can include a survey that captures patient's self-assessment of the patient's health. Medical study instrument information can include a data derived from a questionnaire such as question-answer pairs. In some implementations, when answers to the questions of a question-answer pair are populated by a patient, a patient score based on one or more of the question-answer pairs can be determined. The patient score can include a pre-treatment score, a post-treatment score, or the like.

Accordingly, using the one or more devices 120, a patient can transmit information representing patient attributes such as the patient's name, age, sex, social security number, address, or medical history data to the data marketplace computer 110. In some implementations, a patient, or other client-side user, can use a device such as the mobile device 120a to input or select patient information for transmission to the data marketplace computer 110. In some implementations, the one or more devices 120 can receive a request 126 for patient information from the data marketplace computer 110, and in response to the request, the patient device can provide patient information 121 to the data marketplace computer 110 using one or more of the devices 120. For example, a desktop computer 120b or server computer 120c of a medical services provider, medical data aggregator, or the like can receive a request from the data marketplace computer 110 for patient medical data and then the patient medical data can be provided by the desktop computer 120b or server computer 120c to the data marketplace computer.

In some implementations, an explicit request for patient information need not be received by any device 120 in order for the device 120 to provide patient information to the data marketplace computer 110. Instead, a device 120 such as a server 120c can be configured to periodically provide patient information to the data marketplace computer 110. Such periodic transmissions can be triggered by execution of one or more data transmission rules that are defined in a variety of different ways. For example, data transmission rules can be periodic and defined based on periods of time such as nightly, weekly, monthly, or the like. Data transmission rules can also be defined based on data size. For example, when data size of newly acquired patient information satisfies a predetermined threshold, then transmit the newly acquired patient information.

A determination of whether patient information is newly acquired can be determined based on a data flag associated with the patient information on the device 120, or another computer, that indicates whether the patient information has been transmitted to the data marketplace computer. In some implementations, the data flag can be initialized to a first value such as zero when the patient information is acquired by a user device and then toggled to a second value such as one when the patient information is transmitted to a data market place server. Accordingly, any data that is associated with a flag and has a flag set to the first value such as zero can be determined to be newly acquired and the aggregate size of the newly acquired patient information that is associated with the set flag can be evaluated against a threshold defined by a data transmission rule.

In some implementations, patient information can be transmitted without an explicit request for patient information and using data transmission rules to efficiently manage network bandwidth based on current network bandwidth. For example, a device 120 can monitor current network bandwidth usage and if current utilization of network bandwidth fails to satisfy a predetermined threshold, then the device 120 can begin transmitting newly acquired patient information to the data marketplace computer 110.

The data marketplace computer 110 includes an application programming interface (API) 111, a report generation unit 112, a data marketplace search engine 113, a patient information aggregation unit 114, a patient compensation unit 115, and a patient registry 116. The API 111 is capable of receiving the patient information 121. The API 111 can provide the patient information 121 as an input to the patient information aggregation unit 114.

The patient information aggregation unit 114 is capable of receiving the patient information 121 and processing the patient information 121 to de-identify the patient information 121. For example, the patient information aggregation unit 114 can remove identity information of the patient, such as information relating to the name, social security number, and address of the patient that provided the patient information 121. In the shown implementation, the resulting de-identified patient information may only include the patient's sex, age, and medical history data.

In some implementations, the patient information aggregation unit 114 implements machine learning techniques such as, neural networks, Bayesian algorithms, or the like to de-identify the identity information of the patient to be removed. For example, the patient information aggregation unit 114 can use a machine learning model that has been trained to detect information that is formatted in a particular manner. For example, in some implementations, a machine learning model can be trained to detect information that includes information formatted in the form of social security number. Such training can occur by first training the model using trained data items having a social security number associated with a first label and training data items that do not include social security numbers associated with a second label. The model can be used to process the trained data items and then a user can adjust the parameters of the model based on a comparison of the output data generated by the model based on the model's processing of a training data item to the label for the training data item. The model can be trained until outputs generated by the machine learning model begin to approach the label for the training data item processed to generate the output.

The patient information aggregation unit 114 can search patient information identified by the machine learning model as including sensitive information such as a name, social security number, address, or the like in order to identify the sensitive information and then delete the sensitive information from the patient information. Then, the de-identified information 121a can be assigned a unique wallet identifier and stored in the patient registry 116. The unique wallet identifier can include a string of numbers, a string of alphanumeric characters, a public key associated with a cryptocurrency wallet, or any other data string that can be used to uniquely identified an account or digital wallet. However, it is noted that while a machine learning model can be trained to screen patient information to identify particular types of sensitive patient information that is present in a particular instance of patient information, the present disclosure is not so limited. Instead, all incoming patient information can have non-machine learning model filters applied to search text of the patient information to identified information formatted in one or more sensitive information formats for deletion. However, use of the machine learning filter here allows for intelligent filtering of incoming information to enable efficient removal of sensitive information using load balancing techniques. That is—incoming patient information identified has having a first type of sensitive information such as social security number can be sent to a first sensitive information removal module and different patient information identified as having a second type of sensitive information such as an address can be sent to a second sensitive information removal module.

In the same, or other, implementations, the patient information 121 can be provided in a plurality of fields, in which at least some of the fields are tagged, in advance, for de-identification. For example, the patients social security number can be provided in a field designated for social security number input, and the field can be tagged by the one or more patient devices 120 to indicate that the field includes identification data. In such instances, the patient information aggregation unit 114 can use machine learning techniques or non-machine learning techniques to identify the tagged fields and remove the information in the tagged fields from the patient information 114.

Once the patient information aggregation unit 114 is de-identified, patient information can assign a unique wallet identifier to the de-identified patient information 114, the patient information aggregation unit 114 transmits the de-identified patient information 121a to the patient registry 116 for storage. The patient information aggregation unit 114 can add an entry to a table stored in the patient compensation unit 115 that maps identifiable patient information such as a social security number, business name, product identifier, or the like to the unique wallet identifier. In some implementations, this information can be securely stored in storage device accessible to the patient compensation unit 115. In some implementations, this data can be encrypted and the encryption keys may not be transmitted across any networks and remain on the data marketplace compute 110.

The data marketplace search engine 113 is capable of receiving the request 122 for patient information from a vendor device 160. The vendor device 160 can be any type of computing device, such as a smartphone, a smartwatch, a tablet computer, a laptop computer, a desktop computer, a server computer, or any other type of computer. In some implementations, a vendor can use the vendor device 160 to request patient information related to each patient that has been diagnosed with a certain disease or undergone a treatment within a period of time such as the last five years. In some implementations, the vendor can input data such as a query, query parameters, or both, that describes a limit on search results that are to be received. For example, the vendor can input keywords, a numerical value indicating total number of records requested, or a combination of both. In other implementations, the data marketplace computer 110 can act in conjunction with the vendor or independently of the vendor to limit the amount of search results that are to be returned in response to a request from a vendor. For example, the data marketplace computer 110 can generate a query, query parameters, or both, to limit the search results received. In some implementations, the generated query can be the only query used to limit search results or the generated query can include a revised or refined query that supplements or refines query parameters input by the vendor. The vendor device 160 can send, to the data marketplace computer 110 and through the network 140, data representing a request 122 (e.g., a query, query parameters, or both) for patient information.

In some implementations, the request 122 can be received by the API 111. The API 111 can provide the request 122 for patient information to the data marketplace search engine 113. The data market place search engine 113 can generate a query 122a based on the request and execute the query 122a against the patient registry 116. The data marketplace search engine 113 can access the patient registry 116 to identify patient information stored in the patient registry 116 that satisfies the request 122. For example, if the request 122 indicates a call for all patients that were diagnosed with chronic kidney disease in the last five years, the data marketplace search engine can access the patient registry 116 to identify all patient information corresponding to patients that were diagnosed with chronic kidney disease in the last five years.

In some implementations, the data marketplace search engine 113 can generate a query 122a based on the received request and execute the query 122a against the patient registry 116. By way of example, the query 122a can be a structured query such as an SQL query. In some implementations, the data marketplace search engine can use word matching techniques to identify a subset of patient information in the patient registry 116 that is responsive to the query 122a and satisfies the request 122. For example, if the request 122 is for a set of patients diagnosed with chronic kidney disease, the data marketplace search engine 113 can generate a query 122a or other search request that identifies, in the patient registry 116, a subset of patient information for patients that have medical history data that includes the words “chronic kidney disease” and words associated with “chronic kidney disease” such as “kidney disease”; “CKD”; and similar words, including corresponding words in other languages. Once the data marketplace search engine 113 identifies the patient information 122b that is responsive to the query 122a and that satisfies the request 122, the data marketplace search engine 113 can provide the identified patient information 122b to the API 111 (or other functional module) that is configured to transform the identified patient information into the list of de-identified patient information 124. In some implementations, the data market place search engine 113 may process the search results into obfuscated patient profiles for each search result. Neither the search results 122b or the obfuscated patient profiles include the unique wallet identifiers corresponding to the de-identified patient information in the search results 122b. The API 111 can then provide the list of de-identified patient information 124 to the vendor device 160 as a response to the request 122. In some implementations, the API may also modify the inputs such as 122b it receives to package the information for transmission as a message such as 124 to a device such as vendor device 160 so that the message 124 includes certain protocol formats necessary for transmission.

The list of de-identified patient information 124 provided by the data marketplace 110 and to the vendor device 160 can include an obfuscated profile for each patient that satisfies the request 122. For example, the obfuscated profile can include the sex, age, and medical history data of each patient that satisfies the request 122. Medical history data can include, for example, outcome scores for a patient. When the data representing the list of de-identified patient information 124 is received by the vendor device 160, the vendor device 160 is caused to render, on a display of the vendor device 160, a first user interface 124a having one or more graphical representations representing the received list of obfuscated patient profile information 124. For example, the first user interface 124a can display an obfuscated profile of each patient that satisfies the request 122. In the example of FIG. 1, the first user interface 124a includes a “Market Place Search Results” list that includes an obfuscated profile for a plurality of patients having patient information stored by the data marketplace computer 110 that satisfy the request 122.

The one or more graphical representations of the first user interface 124a can include user-selectable icons that allow a user of the vendor device 160 to select one or more obfuscated patient profiles provided for display using the user interface 124a. Once the user of the vendor device 160 has made a set of one or more selections, the vendor device 160 can transmit, to the data marketplace computer 110 through the network 140, selection data 123 representing the set of user's selections. The data marketplace computer 110 can obtain and transmit, based on the received selection data 123, summary data 125 representing a summary of the medical history of the patients that corresponds to the obfuscated profiles selected using the first user interface 124a. In response to receiving the summary data 125, the vendor device 160 is caused to render, on the display of the vendor device 160, a second user interface 125a having one or more graphical representations representing the summary of the medical history.

The summary data 125 can be generated by the report generation unit 112. The report generation unit 112 can receive the selection data 123 and generate summary data 125 that summarizes the patient information of the selected one or more patients identified by the selection data. For example, the report generation unit 112 can receive the selection data 123 and access the patient registry 116 to locate a subset of patient information from the patient registry 116 that corresponds to the selection data 123. Accessing the patient registry 116 can include generating, by the report generation unit 112 or other functional unit such as a search engine, a query 123a based on selection data 123. The report generation unit 112, or other search engine, can execute the query 123a against the patient registry 116 to obtain a subset of patient information 123b for use in generating the summary data 125.

The report generation unit 112 can then generate summary data 125 using the subset of patient information 123b, with the summary data 125 representing a summary of the patient information for a patient associated with the selection data 123. In one implementation, the summary data 125 includes a cumulative summary in which the patient information of patients identified by the selection data 125 are included in one summary. In one implementation, the summary data 125 includes an individual summary of patient information for each patient identified by the selection data 125. The report generation unit 112 transmits the summary data 125 to the API 111 for forwarding to the vendor device 160.

A computer 130 can be used maintain a data storage device 132 that stores one or more user accounts such as digital wallet 132a for a patient. The digital wallet 132a can be a portion of allocated memory of an electronic device such as computer 130 that stores information representing a unit of value owned, or otherwise associated with, the owner of the digital wallet 132a. An owner of the digital wallet 132a can use the units of value represented by the information stored in the wallet to execute electronic transactions.

In some implementations, the digital wallet 132a includes a financial account of a user that is specific to the digital wallet 132a. In some implementations, each digital wallet can correspond to a particular and unique wallet identifier. Additionally, or alternatively, a user can integrate one or more of their financial accounts corresponding to other sources, such as a bank account, with the digital wallet 132a. The user can use the digital wallet 132a to make purchases or obtain redemptions. For example, the user can use the digital wallet 130 to make in store purchases or on-line purchases. The digital wallet 132a is capable of receiving compensation data 127 from the digital marketplace computer 110, and updating the accounts corresponding to the digital wallet 132a in accordance with the compensation data 127. However, the digital wallet 132a is not limited to wallets tied to traditional bank accounts having a value unit denominated in one or more particular country's currency. Instead, the digital wallet 123a can store information representing other types of units of value. In some implementations, for example, the digital wallet 132a can be a cryptocurrency wallet storing data representing a unit of value of one or more cryptocurrencies. In some implementations, for example, the digital wallet can be a wallet that stores data representing a unit of value of one or more rewards programs. Yet other types of digital wallets that store, and facilitate transactions using, information representing other units of value also fall within the scope of the present disclosure.

In some implementations, the rewards can be existing cryptocurrencies such as bitcoin (BTC) or ethereum (ETH). In other implementations, the cryptocurrencies can include a custom cryptocurrency token designed using a cryptocurrency protocol such as ETH for a particular application such as for use as a loyalty rewards. Though BTC and ETH are listed here, they are merely examples of existing cryptocurrencies or blockchain protocols that can be used as a unit of value to compensate a user. Other cryptocurrencies can be also be used, such as a custom built cryptocurrency and/or blockchain, to facilitate the purposes described herein.

The patient compensation unit 115 is capable of receiving the selection data 123 and generating compensation data 127 for each selected patient. In some implementations, the patient compensation unit 115 determines compensation by accessing and evaluating a patient profile for each patient identified by the selection data 123. For example, the patient compensation unit 115 can access a unique wallet identifier associated with the de-identified patient information stored in the patient registry 116 that corresponds to the selection data 123. This unique wallet identifier can be stored in associated with the de-identified patient information in the patient registry and not be previously transmitted to the vendor device 160.

In some implementations, the patient profiles may be stored in the patient registry 116 and each patient profile can include a unique wallet identifier corresponding to a digital wallet for the patient whose data was used to generate the de-identified data. In such implementations, the patient compensation unit 115 can access the patient registry 116 to obtain patient profiles for each patient identified by the selection data 123 and evaluate the obtained patient profiles. In other implementations, the patient profiles may be stored in a separate and secure (e.g., encrypted) patient profile database. In such implementations, the patient compensation unit 115 can access the patient profile database to obtain patient profiles for each patient identified by the selection data 123, and obtain the unique wallet identifier associated with the profile that correspond to the selection data 123.

A patient profile can include one or more of: data representing a unique wallet identifier, a score indicating quality of the patient information, data that describes a form of compensation for the patient, data that describes a patient's level of compliance, data describing a volume of patient surgeries, data describing procedure types that were performed on the patient, data describing patient reported outcome measures (PROMs) used (for example, a survey that captures a patient's self-assessment of health), type of data stored in the patient registry database for the patient, an amount of data provided to the patient registry database by the patient, and a utility score indicating a usefulness of the data stored in the patient database registry for the patient. Compensation can take the form of any type of currency having monetary values, such as fiat currency, commodity currency, cryptocurrency, gift cards, airline miles, and so forth. Once a compensation is determined for a selected patient, the patient compensation unit 115 provides the compensation data 127 representing the determined compensation to the API 111. The API 111 can generate a payment transaction based on (i) the selection data 123 and (ii) evaluation of a patient profile for each patient identified by the selection data 123. For example, the API 111 can generate a payment transaction that includes the unique wallet identifier determined based on an evaluation of the patient profile for each patient identified by the selection data 123. The API 111 can initiate a payment transaction to a digital wallet such as digital wallet 132a for each patient identified by the selection data 123.

Aspects of the present disclosure describe interactions between multiple client-side devices such as devices 120 and device 160 and the data marketplace computer 110. In addition, aspects of the present disclosure describe interactions between the data marketplace computer 110 and the devices 120, the device 160, and the device 130. In the examples above, each of the interactions includes requests and information routed through the API 111 of the data marketplace computer. The API 111 can provide a central processing node implemented by a combination of software such as middleware and hardware components that can intelligently manage data interactions between the aforementioned computing devices. By way of example, this central processing node can track interactions, timestamp interactions, and help to ensure data integrity such that as the patient registry 116 is being populated with new patient data—requests for patient information are receiving responses that reflect newly updated data.

However, the present disclosure need not be so limited. For example, in some implementations, the data marketplace computer 110 can be implemented without API 111 of the data marketplace computer 110. In such implementations, each processing module of the data marketplace computer 110 can be configured to receive requests from other devices and generate responses that are routed to the corresponding other devices. In such implementations, the processing modules may be configured to communicate with each other as distributed agents to ensure data integrity across transactions.

The system 100 described above is configured perform the functionality described in this specification both above and below. In addition, the system 100 can be configured to perform any of the functionality described by the medical storefront as described in U.S. Pub. No. 2011/0161105, which is herein incorporated by reference in its entirety.

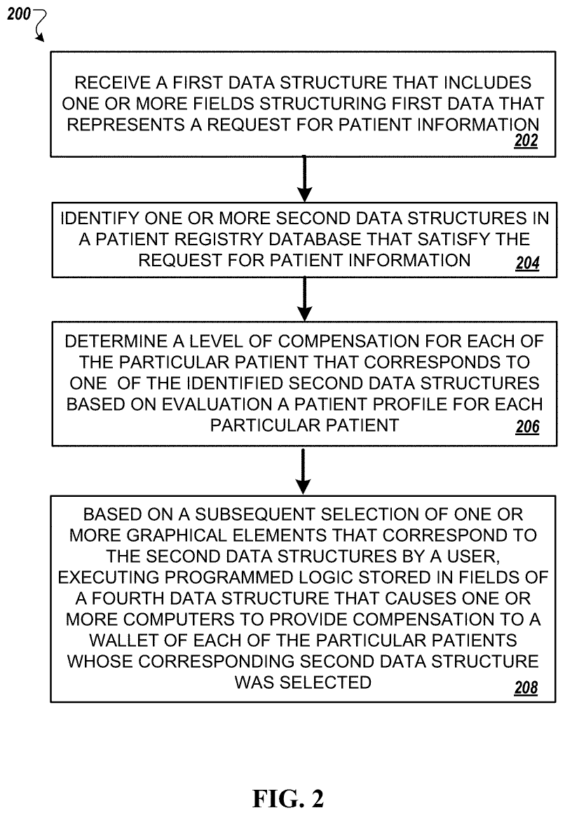

FIG. 2 is a flowchart of an example of a process 200 for using databases, data structures, and data protocols to execute an electronic transaction in a data marketplace. For illustrative purposes, the method 200 is described as being performed by the system 100 discussed earlier with reference to FIG. 1. The method 200 includes: receiving a first data structure that includes one or more fields structuring first data that represents a request for patient information (block 202); identifying one or more second data structures in a patient registry database that satisfy the request for patient information (block 204); determining a level of compensation for each of the particular patient that corresponds to one of the identified second data structures based on evaluation a patient profile for each particular patient (block 206); and based on a subsequent selection of one or more graphical elements that correspond to the second data structures by a user, executing programmed logic stored in fields of a fourth data structure that causes one or more computers to provide compensation to a wallet of each of the particular patients whose corresponding second data structure was selected (block 208).

At block 202, the data marketplace computer 110 receives the request 122 for patient information from the vendor device 160. In some implementations, the patient information includes health records, medical instrument information, outcome information, or a combination of them.

At block 204, the data marketplace search engine 113 accesses the patient registry 116 to identify patient information 121 that satisfies the request 122. In some implementations, each of the one or more second data structures correspond to a particular patient and include fields structuring data that represents patient information for the particular patient. In some implementations, identifying the one or more second data structures includes obtaining, by the patient information aggregation unit 114, the first data from the first data structure. In some implementations, identifying the one or more second data structures includes generating, by the data marketplace search engine 113, a search query that includes the obtained first data. In some implementations, identifying the one or more second data structures includes executing, by the data marketplace search engine 113, the generated search query against the patient registry 116.

At block 206 determines compensation for each patient that has provided their patient information to the data marketplace computer 110 by accessing the patient registry 116 and evaluating a patient profile for each patient based on the patient information in the patient registry. In some implementations, the data representing the patient profile for one of the particular patients includes one or more of: (i) data representing a score indicating quality of the patient information, (ii) data that describes a form of compensation for the patient, (iii) data that describes a patient's level of compliance (iv) data describing a volume of patient surgeries, (v) data describing procedure types that were performed on the patient, (vi) data describing patient reported outcome measures (PROMs) used, (vii) type of data stored in the patient registry database for the patient, (viii) an amount of data provided to the patient registry database by the patient, and (ix) a utility score indicating a usefulness of the data stored in the patient database registry for the patient.

At block 208, the patient compensation unit 115 receives the selection data 123 transmitted by the vendor device 160 and determines which patients to compensate based on the selection data 123. The patient compensation unit 115 generates compensation data 127 representing the determined compensation of a selected patient to the API 111 for forwarding to the digital wallet 130 of the patient.

In some implementations, the method 200 includes generating, by the one or more computers of the data marketplace, a fifth data structure that includes fields structuring rendering data that, when executed by the user device, causes the user device to display a graphical record, for each particular second data structures of the one or more identified second data structures, wherein the graphical record displays at least a portion of the patient information for the particular patient that corresponds to the particular second data structure; and providing, by the one or more computers of the data marketplace, the fifth data structure that includes the fields structuring the rendering data to the user device. For example, as indicated previously with reference to FIG. 1, the data marketplace computer 110 can receive the request 122 for patient information and, in response, can transmit data representing a list of de-identified patient information 124 to the vendor device 160 that satisfies the request 122. The list of de-identified patient information 124 can include a de-identified profile for each patient that satisfies the request 122. When the data representing the list of de-identified patient information 124 is received by the vendor device 160, the vendor device 160 can be caused to render, on a display of the vendor device 160, a first user interface 124a having one or more graphical representations representing the received list of de-identified patient information 124. The one or more graphical representations of the first user interface 124a can include user-selectable icons that allow the user of the vendor device 160 to select one or more patients (that is, the patient profile of each of the one or more patients) in the list of de-identified patient information 124 using the user interface 124a.

In some implementations, the method 200 includes receiving, by one or more computers of a data marketplace and from a user device, a sixth data structure that includes one or more fields structuring second data that represents a user's selection of one or more of the graphical record displays that each corresponds to one of the identified second data structures. For example, as indicated previously with reference to FIG. 1, the data marketplace computer 110 can receive selection data 123 representing the user's selections.

In some implementations, the method 200 includes generating, by the one or more computers of the data marketplace, a report that summarizes the second data of at least one of the one or more identified second data structures that correspond to the one or more second data structures having second data that was selected by the user; and providing, by the user one or more computers, the generated report to the user without providing the second data of the one or more identified second data structures. As indicated previously with reference to FIG. 1, the report generation unit 112 can receive the selection data 123 and generate summary data 125 summarizing the medical history data of the selected one or more patients.

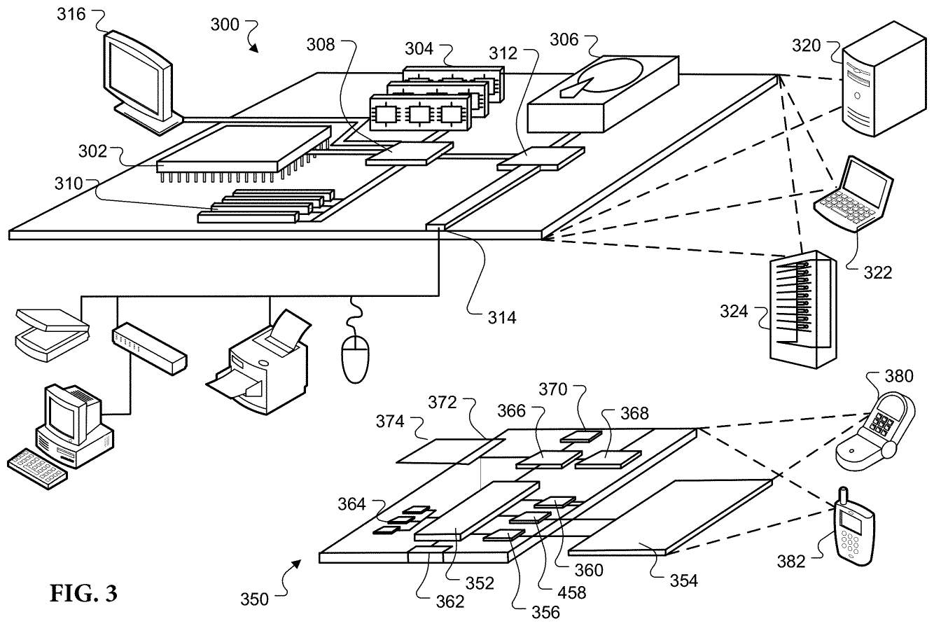

FIG. 3 is a block diagram of system components that can be used to implement a system for using databases, data structures, and data protocols to execute an electronic transaction in a data marketplace.

Computing device 300 is intended to represent various forms of digital computers, such as laptops, desktops, workstations, personal digital assistants, servers, blade servers, mainframes, and other appropriate computers. Computing device 350 is intended to represent various forms of mobile devices, such as personal digital assistants, cellular telephones, smartphones, and other similar computing devices. Additionally, computing device 300 or 350 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 300 includes a processor 302, memory 304, a storage device 306, a high-speed interface 308 connecting to memory 304 and high-speed expansion ports 310, and a low speed interface 312 connecting to low speed bus 314 and storage device 306. Each of the components 302, 304, 306, 308, 310, and 312, are interconnected using various busses, and can be mounted on a common motherboard or in other manners as appropriate. The processor 302 can process instructions for execution within the computing device 300, including instructions stored in the memory 304 or on the storage device 308 to display graphical information for a GUI on an external input/output device, such as display 316 coupled to high speed interface 308. In other implementations, multiple processors and/or multiple buses can be used, as appropriate, along with multiple memories and types of memory. Also, multiple computing devices 300 can be connected, with each device providing portions of the necessary operations, e.g., as a server bank, a group of blade servers, or a multi-processor system.

The memory 304 stores information within the computing device 300. In one implementation, the memory 304 is a volatile memory unit or units. In another implementation, the memory 304 is a non-volatile memory unit or units. The memory 304 can also be another form of computer-readable medium, such as a magnetic or optical disk.

The storage device 308 is capable of providing mass storage for the computing device 300. In one implementation, the storage device 308 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 304, the storage device 308, or memory on processor 302.

The high speed controller 308 manages bandwidth-intensive operations for the computing device 300, while the low speed controller 312 manages lower bandwidth intensive operations. Such allocation of functions is exemplary only. In one implementation, the high-speed controller 308 is coupled to memory 304, display 316, e.g., through a graphics processor or accelerator, and to high-speed expansion ports 310, which can accept various expansion cards (not shown). In the implementation, low-speed controller 312 is coupled to storage device 308 and low-speed expansion port 314. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 300 can be implemented in a number of different forms, as shown in the figure. For example, it can be implemented as a standard server 320, or multiple times in a group of such servers. It can also be implemented as part of a rack server system 324. In addition, it can be implemented in a personal computer such as a laptop computer 322. Alternatively, components from computing device 300 can be combined with other components in a mobile device (not shown), such as device 350. Each of such devices can contain one or more of computing device 300, 350, and an entire system can be made up of multiple computing devices 300, 350 communicating with each other.

The computing device 300 can be implemented in a number of different forms, as shown in the figure. For example, it can be implemented as a standard server 320, or multiple times in a group of such servers. It can also be implemented as part of a rack server system 324. In addition, it can be implemented in a personal computer such as a laptop computer 322. Alternatively, components from computing device 300 can be combined with other components in a mobile device (not shown), such as device 350. Each of such devices can contain one or more of computing device 300, 350, and an entire system can be made up of multiple computing devices 300, 350 communicating with each other.

Computing device 350 includes a processor 352, memory 364, and an input/output device such as a display 354, a communication interface 366, and a transceiver 368, among other components. The device 350 can also be provided with a storage device, such as a micro-drive or other device, to provide additional storage. Each of the components 350, 352, 364, 354, 366, and 368, are interconnected using various buses, and several of the components can be mounted on a common motherboard or in other manners as appropriate.

The processor 352 can execute instructions within the computing device 350, including instructions stored in the memory 364. The processor can be implemented as a chipset of chips that include separate and multiple analog and digital processors. Additionally, the processor can be implemented using any of a number of architectures. For example, the processor 310 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 350, such as control of user interfaces, applications run by device 350, and wireless communication by device 350.

Processor 352 can communicate with a user through control interface 358 and display interface 356 coupled to a display 354. The display 354 can be, for example, a TFT (Thin-Film-Transistor Liquid Crystal Display) display or an OLED (Organic Light Emitting Diode) display, or other appropriate display technology. The display interface 356 can comprise appropriate circuitry for driving the display 354 to present graphical and other information to a user. The control interface 358 can receive commands from a user and convert them for submission to the processor 352. In addition, an external interface 362 can be provide in communication with processor 352, so as to enable near area communication of device 350 with other devices. External interface 362 can provide, for example, for wired communication in some implementations, or for wireless communication in other implementations, and multiple interfaces can also be used.

The memory 364 stores information within the computing device 350. The memory 364 can be implemented as one or more of a computer-readable medium or media, a volatile memory unit or units, or a non-volatile memory unit or units. Expansion memory 374 can also be provided and connected to device 350 through expansion interface 372, which can include, for example, a SIMM (Single In Line Memory Module) card interface. Such expansion memory 374 can provide extra storage space for device 350, or can also store applications or other information for device 350. Specifically, expansion memory 374 can include instructions to carry out or supplement the processes described above, and can include secure information also. Thus, for example, expansion memory 374 can be provide as a security module for device 350, and can be programmed with instructions that permit secure use of device 350. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 364, expansion memory 374, or memory on processor 352 that can be received, for example, over transceiver 368 or external interface 362.

Device 350 can communicate wirelessly through communication interface 366, which can include digital signal processing circuitry where necessary. Communication interface 366 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 368. In addition, short-range communication can occur, such as using a Bluetooth, Wi-Fi, or other such transceiver (not shown). In addition, GPS (Global Positioning System) receiver module 370 can provide additional navigation- and location-related wireless data to device 350, which can be used as appropriate by applications running on device 350.

Device 350 can also communicate audibly using audio codec 360, which can receive spoken information from a user and convert it to usable digital information. Audio codec 360 can likewise generate audible sound for a user, such as through a speaker, e.g., in a handset of device 350. Such sound can include sound from voice telephone calls, can include recorded sound, e.g., voice messages, music files, etc. and can also include sound generated by applications operating on device 350.

The computing device 350 can be implemented in a number of different forms, as shown in the figure. For example, it can be implemented as a cellular telephone 380. It can also be implemented as part of a smartphone 382, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

1. A system for executing a transaction in a data marketplace, the system comprising:
one or more data processing apparatus; and
one or more computer-readable storage devices having stored thereon instructions that, when executed by the one or more data processing apparatus, cause the one or more data processing apparatus to perform operations comprising:
receiving, by one or more computers of a data marketplace and from a user device, a first data structure that includes one or more fields structuring first data that represents a request for patient information;
identifying, based on the receipt of the first data that represents the request for patient information, one or more second data structures in a patient registry database that satisfy the request for patient information that is represented by the first data, wherein each of the one or more second data structures (i) correspond to a particular patient and (ii) include fields structuring second data that represents patient information for the particular patient;
determining, by the one or more computers of the data marketplace, a level of compensation for each of the particular patients, based on an evaluation of third data structured by fields of a plurality of third data structures, wherein each third data structure includes fields structuring data representing a patient profile for one of the particular patients; and
based on a determination, by the one or more computers of the data marketplace that a subsequent request from the user device selects at least a portion of the second data of at least one of the one or more identified second data structures, executing programmed logic structured by fields of a fourth data structure that causes the one or more computers to provide compensation to a wallet of each of the particular patients that correspond to the at least one or more of the identified second data structures from which at least a portion of the second data was selected, wherein the provided compensation is based on the determined level of compensation.

2. The system of claim 1, the operations further comprising;
generating, by the one or more computers of the data marketplace, a fifth data structure that includes fields structuring rendering data that, when executed by the user device, causes the user device to display a graphical record, for each particular second data structures of the one or more identified second data structures, wherein the graphical record displays at least a portion of the patient information for the particular patient that corresponds to the particular second data structure; and
providing, by the one or more computers of the data marketplace, the fifth data structure that includes the fields structuring the rendering data to the user device.

3. The system of claim 2, the operations further comprising:
receiving, by one or more computers of a data marketplace and from a user device, a sixth data structure that includes one or more fields structuring second data that represents a user's selection of one or more of the graphical record displays that each corresponds to one of the identified second data structures.

4. The system of claim 1, wherein the patient information includes health records, medical instrument information, or outcome information.

5. The system of claim 1, the operations further comprising:
generating, by the one or more computers of the data marketplace, a report that summarizes the second data of at least one of the one or more identified second data structures that correspond to the one or more second data structures having second data that was selected by the user; and
providing, by the user one or more computers, the generated report to the user without providing the second data of the one or more identified second data structures.

6. The system of claim 1, wherein the data representing a patient profile for one of the particular patients includes one or more of: (i) data representing a score indicating quality of the patient information, (ii) data that describes a form of compensation for the patient, (iii) data that describes a patient's level of compliance (iv) data describing a volume of patient surgeries, (v) data describing procedure types that were performed on the patient, (vi) data describing patient reported outcome measures (PROMs) used, (vii) type of data stored in the patient registry database for the patient, (viii) an amount of data provided to the patient registry database by the patient, and (ix) a utility score indicating a usefulness of the data stored in the patient database registry for the patient.

7. The system of claim 1, wherein identifying, based on the receipt of the first data that represents the request for patient information, one or more second data structures in a patient registry database that satisfy the request for patient information that is represented by the first data, wherein each of the one or more second data structures (i) correspond to a particular patient and (ii) include fields structuring data that represents patient information for the particular patient comprises:
obtaining, by the one or more computers of the data marketplace, the first data from the first data structure;
generating, by the one or more computers of the data marketplace, a search query that includes the obtained first data; and
executing, by the one or more computers of the data marketplace, the generated search query against the patient registry database.

8. A method for executing a transaction in a data marketplace comprising:
receiving, by one or more computers of a data marketplace and from a user device, a first data structure that includes one or more fields structuring first data that represents a request for patient information;
identifying, based on the receipt of the first data that represents the request for patient information, one or more second data structures in a patient registry database that satisfy the request for patient information that is represented by the first data, wherein each of the one or more second data structures (i) correspond to a particular patient and (ii) include fields structuring second data that represents patient information for the particular patient;
determining, by the one or more computers of the data marketplace, a level of compensation for each of the particular patients, based on an evaluation of third data structured by fields of a plurality of third data structures, wherein each third data structure includes fields structuring data representing a patient profile for one of the particular patients; and
based on a determination, by the one or more computers of the data marketplace that a subsequent request from the user device selects at least a portion of the second data of at least one of the one or more identified second data structures, executing programmed logic structured by fields of a fourth data structure that causes the one or more computers to provide compensation to a wallet of each of the particular patients that correspond to the at least one or more of the identified second data structures from which at least a portion of the second data was selected, wherein the provided compensation is based on the determined level of compensation.

9. The method of claim 8, the method further comprising;
generating, by the one or more computers of the data marketplace, a fifth data structure that includes fields structuring rendering data that, when executed by the user device, causes the user device to display a graphical record, for each particular second data structures of the one or more identified second data structures, wherein the graphical record displays at least a portion of the patient information for the particular patient that corresponds to the particular second data structure; and
providing, by the one or more computers of the data marketplace, the fifth data structure that includes the fields structuring the rendering data to the user device.

10. The method of claim 9, the method further comprising:
receiving, by one or more computers of a data marketplace and from a user device, a sixth data structure that includes one or more fields structuring second data that represents a user's selection of one or more of the graphical record displays that each corresponds to one of the identified second data structures.

11. The method of claim 8, wherein the patient information includes health records, medical instrument information, or outcome information.

12. The method of claim 8, the method further comprising:
generating, by the one or more computers of the data marketplace, a report that summarizes the second data of at least one of the one or more identified second data structures that correspond to the one or more second data structures having second data that was selected by the user; and
providing, by the user one or more computers, the generated report to the user without providing the second data of the one or more identified second data structures.

13. The method of claim 8, wherein the data representing a patient profile for one of the particular patients includes one or more of: (i) data representing a score indicating quality of the patient information, (ii) data that describes a form of compensation for the patient, (iii) data that describes a patient's level of compliance (iv) data describing a volume of patient surgeries, (v) data describing procedure types that were performed on the patient, (vi) data describing patient reported outcome measures (PROMs) used, (vii) type of data stored in the patient registry database for the patient, (viii) an amount of data provided to the patient registry database by the patient, and (ix) a utility score indicating a usefulness of the data stored in the patient database registry for the patient.

14. The method of claim 8, wherein identifying, based on the receipt of the first data that represents the request for patient information, one or more second data structures in a patient registry database that satisfy the request for patient information that is represented by the first data, wherein each of the one or more second data structures (i) correspond to a particular patient and (ii) include fields structuring data that represents patient information for the particular patient comprises:
obtaining, by the one or more computers of the data marketplace, the first data from the first data structure;
generating, by the one or more computers of the data marketplace, a search query that includes the obtained first data; and
executing, by the one or more computers of the data marketplace, the generated search query against the patient registry database.

15. A computer-readable storage device having stored thereon instructions, which, when executed by data processing apparatus, cause the data processing apparatus to perform operations comprising:
receiving, by one or more computers of a data marketplace and from a user device, a first data structure that includes one or more fields structuring first data that represents a request for patient information;
identifying, based on the receipt of the first data that represents the request for patient information, one or more second data structures in a patient registry database that satisfy the request for patient information that is represented by the first data, wherein each of the one or more second data structures (i) correspond to a particular patient and (ii) include fields structuring second data that represents patient information for the particular patient;
determining, by the one or more computers of the data marketplace, a level of compensation for each of the particular patients, based on an evaluation of third data structured by fields of a plurality of third data structures, wherein each third data structure includes fields structuring data representing a patient profile for one of the particular patients; and
based on a determination, by the one or more computers of the data marketplace that a subsequent request from the user device selects at least a portion of the second data of at least one of the one or more identified second data structures, executing programmed logic structured by fields of a fourth data structure that causes the one or more computers to provide compensation to a wallet of each of the particular patients that correspond to the at least one or more of the identified second data structures from which at least a portion of the second data was selected, wherein the provided compensation is based on the determined level of compensation.

16. The computer-readable storage device of claim 15, the operations further comprising;
generating, by the one or more computers of the data marketplace, a fifth data structure that includes fields structuring rendering data that, when executed by the user device, causes the user device to display a graphical record, for each particular second data structures of the one or more identified second data structures, wherein the graphical record displays at least a portion of the patient information for the particular patient that corresponds to the particular second data structure; and
providing, by the one or more computers of the data marketplace, the fifth data structure that includes the fields structuring the rendering data to the user device.

17. The computer-readable storage device of claim 16, the operations further comprising:
receiving, by one or more computers of a data marketplace and from a user device, a sixth data structure that includes one or more fields structuring second data that represents a user's selection of one or more of the graphical record displays that each corresponds to one of the identified second data structures.

18. The computer-readable storage device of claim 15, the operations further comprising:
generating, by the one or more computers of the data marketplace, a report that summarizes the second data of at least one of the one or more identified second data structures that correspond to the one or more second data structures having second data that was selected by the user; and
providing, by the user one or more computers, the generated report to the user without providing the second data of the one or more identified second data structures.

19. The computer-readable storage device of claim 15, wherein the data representing a patient profile for one of the particular patients includes one or more of: (i) data representing a score indicating quality of the patient information, (ii) data that describes a form of compensation for the patient, (iii) data that describes a patient's level of compliance (iv) data describing a volume of patient surgeries, (v) data describing procedure types that were performed on the patient, (vi) data describing patient reported outcome measures (PROMs) used, (vii) type of data stored in the patient registry database for the patient, (viii) an amount of data provided to the patient registry database by the patient, and (ix) a utility score indicating a usefulness of the data stored in the patient database registry for the patient.

20. The computer-readable storage device of claim 15, wherein identifying, based on the receipt of the first data that represents the request for patient information, one or more second data structures in a patient registry database that satisfy the request for patient information that is represented by the first data, wherein each of the one or more second data structures (i) correspond to a particular patient and (ii) include fields structuring data that represents patient information for the particular patient comprises:
obtaining, by the one or more computers of the data marketplace, the first data from the first data structure;
generating, by the one or more computers of the data marketplace, a search query that includes the obtained first data; and
executing, by the one or more computers of the data marketplace, the generated search query against the patient registry database.
